(19) United States
(12) Patent Application Publication
(10) Pub. No.: US 2013/0253319 A1
(43) Pub. Date: Sep. 26, 2013

(54) METHOD AND SYSTEM FOR ACQUIRING AND ANALYZING MULTIPLE IMAGE DATA LOOPS

(71) Applicant: ULTRASOUND MEDICAL DEVICES, INC., Ann Arbor, MI (US)

(72) Inventors: James Hamilton, Brighton, MI (US); Eric J. Sieczka, Ann Arbor, MI (US); Eric T. Larson, Ann Arbor, MI (US)

(73) Assignee: ULTRASOUND MEDICAL DEVICES, INC., Ann Arbor, MI (US)

(21) Appl. No.: 13/796,126

(22) Filed: Mar. 12, 2013

Related U.S. Application Data
(60) Provisional application No. 61/614,866, filed on Mar. 23, 2012.

(51) Int. Cl. A61B 8/08; A61B 8/00; A61B 5/0402

(52) U.S. Cl. CPC A61B 8/5223; A61B 8/485; A61B 5/0402; A61B 8/0883; A61B 8/4416; A61B 8/469; A61B 8/463
USPC 600/438; 600/437

(57) ABSTRACT

A method and system for acquiring and analyzing multiple image data loops comprising: receiving a set of ultrasound data, characterizing a tissue, collected over a first collection loop and a second collection loop; determining a tissue parameter distribution within the tissue based on the set of ultrasound data and multi-dimension speckle tracking; receiving identification of at least one region of interest represented in the set of ultrasound data in the first collection loop and the second collection loop; measuring a comparative characteristic, in the region of interest, between the first collection loop and the second collection loop; and rendering at least one of the comparative characteristic and the tissue parameter distribution. The system comprises a processor, an analysis engine, and a user interface, and may further comprise an ultrasound scanner. The system is preferably configured to perform the method.

### CROSS-REFERENCE TO RELATED APPLICATIONS

This application claims the benefit of U.S. Provisional Application Ser. No. 61/614,866, filed on 23 Mar. 2012, which is incorporated in its entirety by this reference.

### TECHNICAL FIELD

This invention relates generally to the medical imaging field, and more specifically to an improved method and system for acquiring and analyzing image data loops.

### BACKGROUND

Ultrasound technologies for accurately measuring tissue motion and deformation, such as speckle tracking and issue Doppler imaging, have provided significant advances for applications such as breast elastography and cardiac strain rate imaging. However, clinical impact and widespread use has been limited because the majority of technologies and methods do not adequately facilitate analysis of multiple image data loops, provide limited analyses of tissue parameters over multiple image data loops, and/or are non-ideal due to other factors. Thus, there is a need in the medical imaging field to create an improved method and system for analyzing multiple image data loops. This invention provides such a new and useful system for acquiring and analyzing multiple image data loops.

### BRIEF DESCRIPTION OF THE FIGURES

FIGS. 1-3 are flowcharts of an embodiment of a method for acquiring and analyzing multiple image data loops and variations thereof;

FIG. 4 is a schematic of the system of a preferred embodiment; and

FIGS. 5A-5D depict exemplary embodiments of the method and system.

### DESCRIPTION OF THE PREFERRED EMBODIMENTS

The following description of preferred embodiments of the invention is not intended to limit the invention to these preferred embodiments, but rather to enable any person skilled in the art to make and use this invention.

### 1. Method

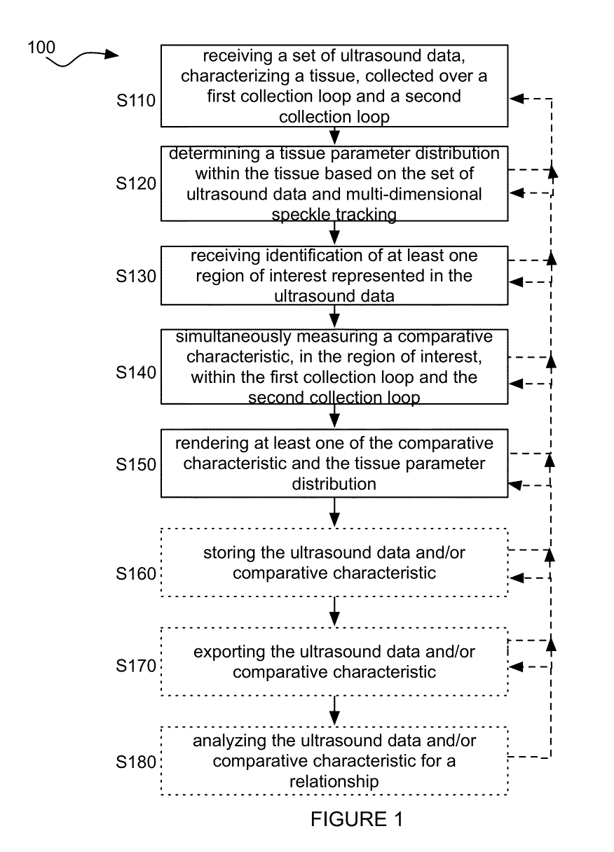

As shown in FIG. 1, a method 100 of an embodiment for acquiring and analyzing image data loops includes: receiving a set of ultrasound data, characterizing a tissue, collected over a first collection loop and a second collection loop S110; determining a tissue parameter distribution within the tissue based on the set of ultrasound data and multi-dimensional speckle tracking S120; receiving identification of at least one region of interest represented in the set of ultrasound data in the first collection loop and the second collection loop S130; measuring a comparative characteristic, in the region of interest, within the first collection loop and the second collection loop S140 based on the region of interest and the tissue parameter distribution; and rendering at least one of the comparative characteristic and the tissue parameter distribution S150. The method can further include storing the ultrasound data and/or comparative characteristic S160, exporting the ultrasound data and/or comparative characteristic S170, and/or analyzing the set of ultrasound data and/or comparative characteristic between collection loops for a relationship S180. The method is preferably used to enable measurement and/or visualization of a tissue, such as cardiac tissue, based on image data collected over different loops or periods of time. For example, the image data can be collected over a cyclical event such as the cardiac cycle, collected over multiple acquisitions of the same tissue at different intervals of time, or collected from tissue of different subjects. Although the method is primarily described herein in regards to ultrasound-based analysis, the image data can be collected over collection loops from any imaging modality suitable for providing markers appropriate for multi-dimension tracking or speckle tracking in the case of ultrasound data. The method is preferably used to characterize cardiac tissue, but can additionally or alternatively be used to characterize other kinds of tissues and structures where comparison of motion characteristics is valuable (e.g., blood vessels, smooth muscle tissue, skeletal muscle tissue).

Step S110 recites receiving a set of ultrasound data, characterizing a tissue, collected over a first collection loop and a second collection loop, which functions to obtain image data loops from which motion characteristics regarding the tissue can be derived and compared. Each loop over which the ultrasound data is collected may capture any suitable tissue event. Preferably, the tissue event is a repeated or repeatable event to facilitate comparisons between tissue events; however, the tissue event may alternatively be a non-repeatable event. For example, the image data can be collected over a cyclical event such as the cardiac cycle or a portion (e.g., subcycle) of a cardiac cycle, collected over multiple acquisitions of the same tissue at different intervals of time (e.g., intermittently, at set time points, continuously), collected over multiple acquisitions of the same tissue in response to a stimulation event, or collected from tissue of different types and/or subjects (e.g., patients). Step S110 preferably includes receiving ultrasound data collected over at least two collection loops, comprising a first collection loop and a second collection loop, but may include receiving ultrasound data collected over less than two collection loops (e.g., a partial loop) or more than two collection loops. In a first example, Step S110 facilitates a stress-echo study, such that the first collection loop comprises a portion (or all) of a cardiac cycle during a rest state, and the second collection loop comprises a portion (or all) or a cardiac cycle during a stress state. In the first example, rest-stress pairs of collection loops may be received for different portions of a cardiac cycle (e.g., systolic cycle, diastolic cycle), or for a complete cardiac cycle. In a second example, Step S110 facilitates a monitoring study, such that the first collection loop comprises at least a portion of a tissue cycle during a first phase of treatment, and the second collection loop comprises a corresponding portion of a tissue cycle during a second phase of treatment. In one variation, the data is received in real-time with collection of the data (e.g., received by a processor coupled to an ultrasound scanner gathering ultrasound data). In another variation, the data is received from a storage device such as a server, cloud storage, computer hard drive, or portable storage medium (e.g., CD, DVD, USB flash drive).

Step S120 recites determining a tissue parameter distribution within the tissue based on the set of ultrasound data and multi-dimensional speckle tracking, which functions to track motion of the tissue over the collection loops as an intermediate step toward generating comparative measurements of tissue motion and/or mechanical function of the tissue between one or more collection loops. Preferably, the tissue parameter distribution is determined across at least the first collection loop and the second collection loop, such that a measurement of a comparative characteristic between the first collection loop and the second collection loop may be made in Step S140. The tissue parameter distribution, however, may be determined across a single collection loop, a portion of a collection loop, and/or more than two collection loops. Additionally, the tissue parameter distribution is preferably determined over an entire ultrasound window, but may alternatively be determined in a portion of an ultrasound window. In an example of Step S120, the tissue parameter is preferably at least one of tissue velocity, tissue displacement, tissue strain, and tissue strain rate, and is determined across both the first collection loop and the second collection loop. In the example, once a region of interest is identified in Step S130, a derivative comparative characteristic, such as ejection fraction (EF) may additionally be measured at Step S140, based on the tissue parameter distribution determined in the example of Step S120 and the identified region of interest from an example of Step S130. In other variations, however, the tissue parameter may be any suitable tissue parameter that may be used to generate a comparative characteristic.

In Step S120, speckle tracking is a motion tracking method implemented by tracking the position of a kernel (section) of ultrasound speckles that are a result of ultrasound interference and reflections from scanned objects. The pattern of ultrasound speckles is substantially similar over small motions, which allows for tracking the motion of the speckle kernel within a region over time. The speckle-tracking algorithm is preferably similar to that described in U.S. Publication No. 2008/0021319, entitled “Method of Modifying Data Acquisition Parameters of an Ultrasound Device” and 2010/0185093, entitled “System and Method for Processing a Real-Time Ultrasound Signal Within a Time Window” which are incorporated in their entirety by this reference, but can alternatively include any suitable speckle-tracking algorithm. Step S120 may be performed one time or multiple times; furthermore, each time Step S120 is performed may involve different or identical parameters of the speckle-tracking algorithm optimized for particular desired characteristic measurements in Step S140.

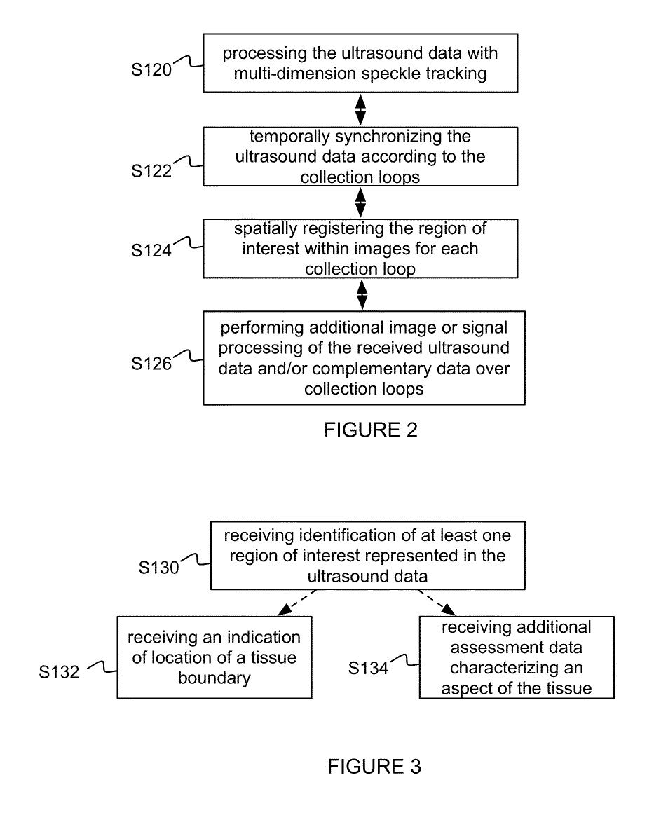

As shown in FIG. 2, the method 100 can further include Step S122, which recites temporally synchronizing the ultrasound data according to the collection loops. Step S122 preferably uses information contained in the post-processed loops (i.e., after applying a speckle-tracking algorithm) and/or additional information such as from electrocardiography (ECG) signals, and functions to temporally synchronize the data and/or define temporal points within a collection loop (e.g., end of systole of a cardiac cycle) to facilitate at least one of Steps S130, S140, and S150. Step S122 may, however, use information contained in pre-processed loops. Preferably, the ultrasound data is temporally synchronized according to tissue motion phase, as opposed to absolute time; however, the ultrasound data may alternatively be temporally synchronized according to any suitable and relevant parameter, including absolute time. In a first example, wherein the first collection loop comprises a portion of a cardiac cycle and the second collection loop comprises a corresponding portion of a cardiac cycle (e.g., for a stress echo study or a patient monitoring study), the first collection loop and the second collection loop may be synchronized by cardiac cycle stages (e.g., diastole, systole). In a second example, wherein the first collection loop comprises a portion of a gait cycle and the second collection loop comprises a corresponding portion of a gait cycle, the first collection loop and the second collection loop may be synchronized by phase of gait. Preferably, Step S122 outputs synchronized image loops or image sequences of the tissue over the collection loops that may facilitate receiving identification of at least one region of interest in Step S130, measuring comparative characteristics in the region of interest in Step S140, and/or are suitable for rendering in Step S150. The ultrasound data may be synchronized using a method similar to that described in U.S. application Ser. No. 13/558,192, entitled “Method and System for Ultrasound Image Computation of Cardiac Events”, which is incorporated in its entirety by this reference; however, the ultrasound data may alternatively be synchronized using any other suitable method. The data can be synchronized, for example, according to a whole cyclical event (e.g., an entire cardiac cycle), a partial cyclical event (e.g., only the systolic cycle in a cardiac cycle), or some combination thereof.

Also shown in FIG. 2, the method 100 may additionally or alternatively include Step S124, which recites spatially registering the region of interest within images for each collection loop. Step S124 functions to mark or co-locate corresponding spatial regions of the ultrasound data, in order to spatially register the ultrasound data and/or to define spatial points within a collection loop or multiple collection loops (e.g., end of systole of a cardiac cycle) to facilitate at least one of Steps S130, S140, and S150. Similarly, the method 100 may include spatially registering any suitable segment of the ultrasound data images, within a portion of a collection loop (e.g., between adjacent frames of a collection loop), such as a tissue boundary (e.g., myocardium) or other appropriate feature detected within an ultrasound image window.

Also shown in FIG. 2, the method 100 may include Step S126, which recites performing additional image or signal processing of the received ultrasound data and/or complementary data over collection loops. For example, the method 100 may include analysis of B-mode features or other speckle tracking properties such as tissue motion parameters (e.g., displacement, velocity, strain, strain rate) or distributions of tissue motion parameters in the received ultrasound data and/or data from other imaging modalities such as electrocardiography modules or magnetic resonance imaging modules. Step S126 may additionally or alternatively include any suitable additional image or signal processing methods.

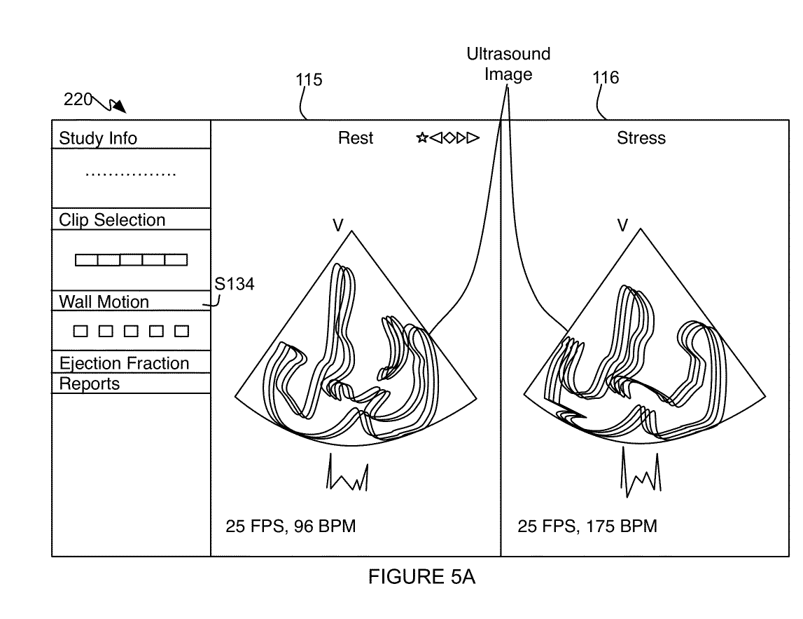

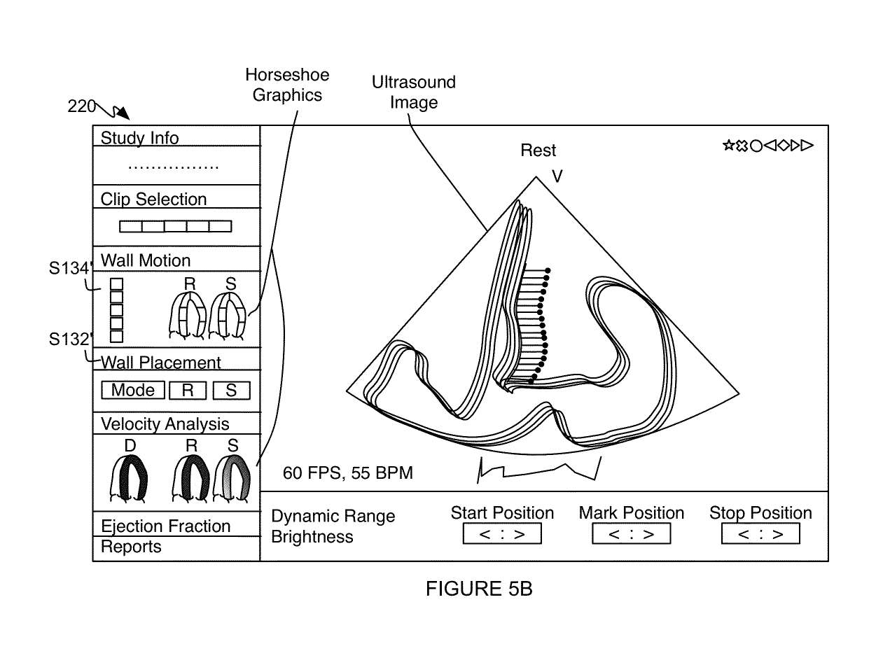

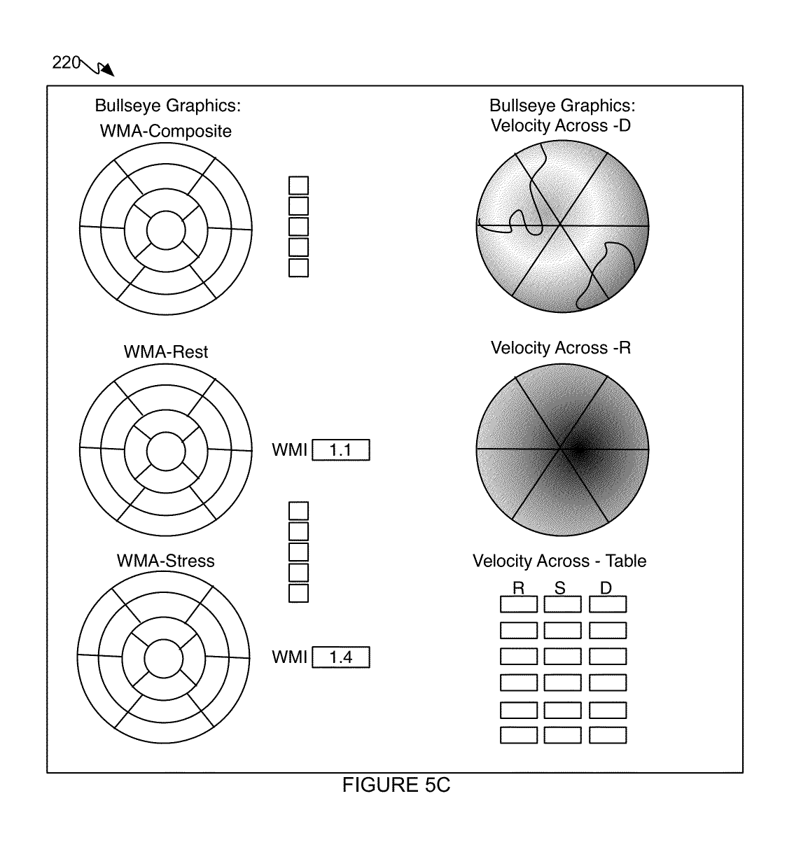

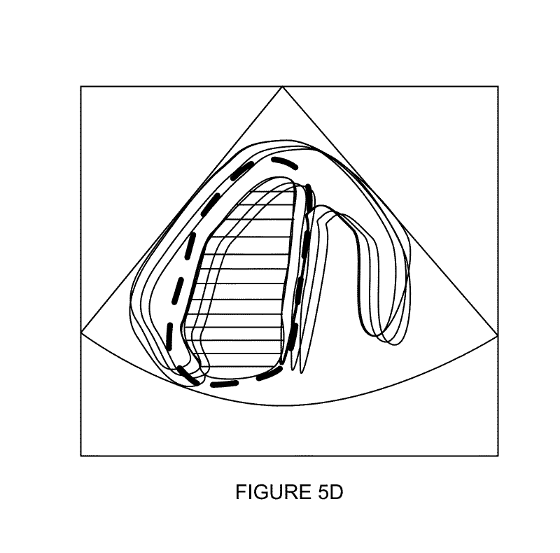

Step S130 recites receiving identification of at least one region of interest represented in the set of ultrasound data in the first collection loop and the second collection loop, which functions to receive information enabling refinement of the processed data, such as to refine the information rendered in Step S150. The identified region of interest preferably describes the tissue location of comparative tissue measurements, for comparisons between multiple collection loops. The identification of the region of interest is preferably received through manual interaction with a user interface, an example of which is shown in FIG. 5B. The user interface is preferably implemented on a computing device with a display, and identification of the region of interest and/or other spatial markers (e.g., tissue boundary) can be manually inputted through any suitable computer interface techniques, such as computer mouse gestures (e.g., clicking points, dragging a mouse cursor) or touch screen gestures. For example, a segment of a region of interest can be identified by a series of clicks or a continuous cursor drag (e.g., creating an outline of the region of interest) with a computer mouse or touch pad. However, the region of interest can additionally or alternatively be identified through automated means (e.g. algorithmically based on previously identified areas representing regions of interest or by boundary detection) or any other suitable process. The region of interest may be identified across multiple portions of ultrasound data or a collection group by manual user input, may be identified once by user input and then tracked through multiple portions of the ultrasound data automatically, or may be identified in a fully automated manner.

As shown in FIG. 3, the method 100 may additionally or alternatively include interacting with the processed data in any other suitable manner. In a first variation, the method 100 may include Step S132, which recites receiving an indication of location of a tissue boundary. In one example of Step S132, the tissue boundary can be indicated in a manner similar to identification of a region of interest in Step S130. In another example of Step S132, the tissue boundary can be indicated by the region of interest in Step S130 coupled with speckle tracking tissue motion data from Step S120. In yet another example of Step S132, the tissue boundary can additionally or alternatively be refined or fine-tuned based on input of information from morphological image processing, and/or complementary data from another imaging modality (e.g., magnetic resonance imaging, computed tomography) across one image frame, a partial collection loop, an entire collection loop, and/or multiple collection loops. The additional information can supplement or replace the information obtained in the speckle-tracking algorithm in Step S120. In one specific example, the location of the myocardium position in the ultrasound images can be refined at the start and end of systole to optimize ejection fraction (EF) measurements and/or velocity measurements.

Also shown in FIG. 3, in a second variation, the method 100 may include Step S134, which recites receiving additional assessment data characterizing an aspect of the tissue. Step S134 functions to facilitate acquisition of additional data to facilitate at least one of Steps S140 and S150. In one example, Step S134 may include receiving a user input of visual or automated wall motion scores, which quantify motion of at least a portion of cardiac tissue (e.g., left ventricular wall). In one example, as shown in FIGS. 5A and 5B, wall motion scores identifying normal motion, hypokinesia, akineasia, and/or dyskinesia may be received for multiple segments cardiac tissue in order to determine a qualitative measure of wall motion. In another example, Step S134 may include receiving known tissue motion constraints (e.g., patient specific tissue features) that facilitate processing of a collection loop or multiple collection loops. However, Step S134 can include receiving any suitable visual and/or automated assessment data to supplement and/or replace any portion of the ultrasound data.

Step S140 of the preferred method recites measuring a comparative characteristic, in the region of interest, within the first collection loop and the second collection loop, which functions to characterize at least the region of interest in regards to tissue motion and/or mechanical function, across multiple collection loops. For example, Step S140 can use any tissue parameter or tissue parameter distribution determined in S120, such as tissue displacement, tissue velocity, tissue strain, tissue strain rate, and/or any suitable parameter(s) in the identified region of interest, within a first collection loop and a second collection loop. In Step S140, the parameter may then be compared between the first collection loop and the second collection loop such as by determining a difference, a distribution of differences, an averaged global difference, or any other suitable comparison in the parameter between the first collection loop and the second collection loop. Step S140 may comprise simultaneously measuring a comparative characteristic, in the region of interest, within the first collection loop and the second collection loop, or may comprise non-simultaneously measuring the comparative characteristic. The comparative characteristic may include any suitable measurement, on a global basis (e.g., over the entire tissue) and/or one or more regional bases (e.g., defined region of interest or boundary). The comparative characteristic may also be derived from the tissue parameter determination from S120, an example of which is measuring and comparing an ejection fraction between two collection loops in S140 based on tissue displacements determined from S120 and regions of interest identified in Step S130. These measurements can be made across multiple contiguous loops (consecutive cycles) from a single acquisition, across multiple acquisitions from a single subject over various time intervals, or across multiple acquisitions from the same subject or different subjects. In one variation, such measurements in Step S140 enable direct assessment of the tissue for comparison between loops, such that the characteristic may be compared between loops (e.g., for diagnostic purposes, for an assessment of treatment success, for a stress-echo study). In another variation, such measurements in Step S140 validate or confirm assessments made visually or through other means. For example, quantification of measurements from speckle tracking may be compared to visual wall motion scoring determined by a visual assessment. Any other suitable comparative characteristic may be alternatively or additionally measured in Step S140.

In an exemplary application in which received ultrasound data is collected over cardiac imaging loops, measurements obtained in Step S140 characterize differences and/or similarities continuously and throughout a cardiac cycle, in peak differences, and/or differences in various cardiac phases (e.g., systole, early diastole, late diastole). For example, movement of the myocardium boundary, identified from the ultrasound data, can be quantified and used to calculate ejection fraction (a common cardiac efficiency measure characterizing a volumetric fraction of blood pumped out of the heart) or other ventricle volumes at particular times in the cardiac cycle, which are useful measures in facilitating diagnoses. In the exemplary application, tissue motion measurements from S120 may be used to determine suitable blood volumes within collection loops. In the exemplary application, differences in tissue velocity distributions across the tissue and/or region of interest may also be measured for comparing the first collection loop and the second collection loop. In another example, tissue boundaries can be measured and used to create an altered B-mode image to enhance visualization of wall or other features, such as to enhance human assessment of wall motion.

Step S150 recites rendering at least one of the comparative characteristic and the tissue parameter distribution, which functions to enable visualization of the ultrasound data and measured comparative characteristics across the collection loops. In an exemplary embodiment of imaging cardiac tissue across cardiac cycles, Step S150 can include rendering ultrasound data in still images and/or video loops, as shown in FIG. 5A, rendering “horseshoe”-shaped graphics, as shown in FIG. 5B, that depict the myocardium (or other cardiac tissue portions) and are color-coded to visualize measurement values, rendering bullseye mappings of regional segments (e.g., left ventricle representation as viewed from the apex) as still images and/or video loops, as shown in, rendering a table of measurement values, as shown in FIG. 5C, and/or any suitable display. The data and characteristics are preferably rendered on a display or user interface of a computing device.

As shown in FIG. 1, the method 100 may further include Step S160, which recites storing at least one of the ultrasound data and comparative characteristic. Step S160 functions to facilitate further analysis of the ultrasound data, and may function to aggregate data from a single patient over time, or from multiple sources such as multiple patients or multiple healthcare institutions. The ultrasound data and/or measured comparative characteristics are preferably stored with corresponding patient data such as demographics or previous data. Aggregating data from a single patient or multiple patients may later facilitate larger-scale analyses included in Step S180. The ultrasound data (raw data or images) and/or corresponding measured comparative characteristics (values or visualizations) can be stored in a database in any suitable storage device, such as a server, cloud storage, computer hard drive, or portable storage medium (e.g., CD, DVD, USB flash drive).

Also shown in FIG. 1, the method 100 may further include other suitable manipulations and treatment of the ultrasound data and/or comparative characteristic. In one variation, the method 100 may include Step S170, which recites exporting at least one of the ultrasound data and comparative characteristic, such as to other data systems. In another variation, the preferred method may include Step S180, which recites analyzing at least one of the ultrasound data and comparative characteristic between collection loops (e.g., a first collection loop and a second collection loop) for a relationship. Step S180 may determine trends and informatics in the patient or across multiple patients, such as with a data mining process or other suitable process. In one variation, Step S180 may further comprise generating an analysis of a single patient based on at least one of the ultrasound data and measured comparative characteristics S185 and/or generating an analysis of multiple patients based on at least one of the ultrasound data and measured comparative characteristics Step S186. Step S185, may for example, include generating an analysis of a patient's response to a treatment based on ultrasound data comprising a series of collection loops that span the treatment period. Step S186 may, for example, include generating an analysis of multiple patients undergoing the same treatment, such that the analysis is used to determine treatment efficacy for a cohort of patients. Other suitable analyses may be performed in Step S180.

The preferred method 100 can include any combination and permutation of the processes described above. Furthermore, as shown in FIG. 1, information derived from any one or more of above processes can be placed in feedback with any other process of the preferred method. For instance, information such as the location of a particular segment (tissue boundary or other region of interest), measured comparative characteristics, or data trends can be fed back into prior processes to modify the algorithms, interactions, measurement process, and/or visualizations to enhance or otherwise modify the overall outcome of the method, such as in an iterative machine learning process.

### 2. System

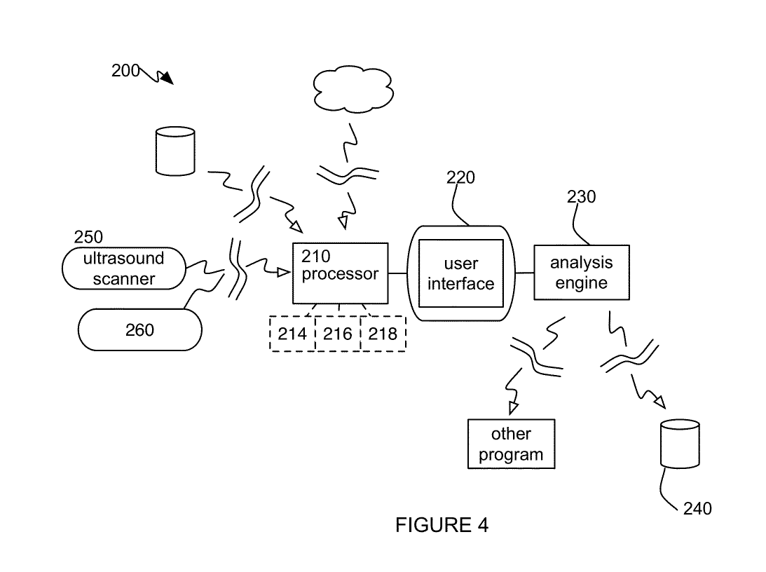

As shown in FIG. 4, a system 200 of the preferred embodiment includes: a processor 210 comprising a first module 214 configured to receive a set of ultrasound data, characterizing a tissue, collected over a first collection loop and a second collection loop, a second module 216 configured to determine a tissue parameter distribution within the tissue based on the set of ultrasound data and multi-dimension speckle tracking, and a third module 218 configured to receive identification of at least one region of interest represented in the set of ultrasound data in the first collection loop and the second collection loop; an analysis engine 230 configured to measure a comparative characteristic, in the region of interest, within the first collection loop and the second collection loop; and a user interface 220, coupled to the processor and the analysis engine, and configured to render at least one of the comparative characteristic and the tissue parameter distribution. The user interface 220 is preferably further configured to render the ultrasound data (e.g., in still images and/or image sequences) and/or the measurement data in representative graphics. The system 200 may further couple to a storage module 240 and/or an ultrasound scanner 250, and may be further configured to couple to an additional imaging module 260.

The processor 210 is configured to couple to the user interface 220, and functions to receive ultrasound data of a tissue, such as cardiac tissue, and to process the ultrasound data using a speckle-tracking algorithm. The processor 210 preferably comprises a first module 214, a second module 216, and a third module 218, as described above; however, the processor 210 may additionally or alternatively comprise any suitable modules configured to receive and process ultrasound data. Preferably, the processor 210, including the first module 214, the second module 216, and the third module 218, is configured to perform a portion of the method 100 described above; however, the processor 210 may be configured to perform any suitable method. The processor 210 is preferably coupled to ultrasound scanning equipment, but can additionally or alternatively be communicatively coupled to a server or other storage device configured to store ultrasound data. The processor 210 preferably performs initial processing of the ultrasound data with a multi-dimension speckle tracking algorithm, and other manipulations of the data such as temporal synchronization and/or spatial registration (e.g., using a fourth module). In a preferred embodiment, the processor 210 performs the processes substantially described in the method 100 described above, but may alternatively perform any suitable process(es).

The analysis engine 230 is configured to couple to the user interface 220, and functions to measure tissue motion comparative characteristics in a region of interest between collection loops. The analysis engine 230 can determine, for example, parameters such as tissue displacement, tissue velocity, strain, and strain rate. The analysis engine 230 may additionally or alternatively be configured to determine any other suitable tissue motion parameter, or to derive parameters based on other tissue parameters. In an exemplary embodiment utilizing ultrasound data of cardiac tissue over cardiac cycles, the analysis engine 230 can determine assessments such as ejection fraction (EF) and blood volume at particular points in a cardiac cycle, based on measurements of tissue displacement and/or tissue velocity. However, the analysis engine 230 may alternatively or additionally determine any suitable comparative characteristic measurements. The analysis engine 230 can additionally or alternatively determine trends in the measured characteristics among data gathered from multiple collection loops, from a single patient, and/or from multiple patients.

The user interface 220 is configured to couple to the processor 210 and the analysis engine 230, and functions to interact with a user (e.g., medical technician or other practitioner) who can manipulate and otherwise interact with the data. For instance, the user interface preferably enables identification of a region of interest and/or tissue boundary and/or visual assessment of characteristics such as wall motion with a wall motion score. The user interface 220 preferably receives input that can be fed back to the processor to enhance or otherwise modify the manner in which the ultrasound data is processed for current and/or future data analyses. The user interface 220 is preferably implanted on a display of a computing device, and can receive input through one or more computer peripheral devices, such as a mouse cursor (e.g., for click selecting and/or dragging), touch screen, motion capture system, or keyboard for data entry.

The user interface 220 is preferably further configured to render ultrasound data, analyses, tissue characteristics, and/or measurements. For instance, in an exemplary embodiment for imaging over collection loops of cardiac cycles, the user interface can render ultrasound data in still images and/or image sequences, render “horseshoe”-shaped graphics that depict the myocardium (or other tissues) and are color-coded to visualize measurement values, render bullseye mappings of regional segments (e.g., left ventricle representation as viewed from the apex) as still images and/or image sequences, render a table of measurement values, and/or any suitable information, as shown in the example of FIGS. 5A-5C.

As shown in FIG. 4, the system 200 may further comprise a storage module 240, such as a server, a cloud, or a hardware device configured to store a database, which stores ultrasound data and/or measured comparative characteristics. The storage module 240 can aggregate data from a single patient over time, or from multiple sources such as multiple patients or multiple healthcare institutions. The ultrasound data and/or measured comparative characteristics are preferably stored with corresponding patient data such as demographics or previous data. The system 200 may also further comprise an ultrasound scanner 250 configured to acquire the set of ultrasound data. In some variations, the system may further be configured to couple to an additional imaging module 260, such as an electrocardiography module, a computed tomography module, a magnetic resonance imaging module, or any other suitably imaging module 260. The imaging module 260 preferably provides supplementary information to facilitate at least one of identification of regions of interest, measurement of a comparative characteristic, and determination of a tissue parameter.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, step, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The method 100 and system 200 of the preferred embodiment can be embodied and/or implemented at least in part as machine configured to receive a computer-readable medium storing computer-readable instructions. The instructions are preferably executed by computer-executable components preferably integrated with the system and one or more portions of the processor and/or analysis engine. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

### 3. Example Implementations

The following example implementations of the method 100 and system 200 are for illustrative purposes only, and should not be construed as definitive or limiting of the scope of the claimed invention. In a first specific example, ultrasound data is collected S110′ from at least one “rest loop” 115 and one “stress loop” 116 of a single cardiac cycle for a stress echo study as shown in FIG. 5A. The data is collected in two-dimensional (2D) views that enable full ventricle measurements comprising a combination of apical 2-chamber, apical 3-chamber, and apical 4-chamber views. The loops are temporally synchronized using ECG signals and motion parameters from speckle tracking S120′. For example, the time for maximum ejection fraction can be used to define end systole of a cardiac cycle. The data in the first specific example is processed for speckle tracking several times, each time having different parameters of the algorithm optimized for the desired characteristic measurements. In the first specific example, the speckle-tracking algorithm can be optimized to locate tissue boundaries (e.g., based on iterated refinements), or to locate contraction of the tissue. Synchronized video loops of the rest and stress loop pairs are then rendered to a user at a user interface. As shown in FIG. 5B, the user enters visual wall motion scores S134′ according to American Society of Echocardiography (ASE) stress echo standards, and interacts with the paired loops (e.g., with a computer mouse cursor or touch screen) to define the boundary of the myocardium and a region of interest on the video loops S132′ and spatially register the video loops S130′. Comparative characteristic measurements of the tissue are then derived comparing values of strains and velocities in the rest and stress loops on both a global basis and a regional basis S140′. These results are presented in horseshoe-shaped graphics that depict the myocardium and are color-coded to visualize the values S150′. As shown in FIG. 5C, the visual wall motion scores and/or other measurement data from the three views are combined to present a three-dimensional representation, such as in a bullseye mapping of the regional segments as viewed from the apex. The bullseye mappings in the first specific example are still images of peak values and/or differences in measurements, and/or video loop (e.g., bullseye image for each frame) synchronized to a corresponding B-mode video loop. As shown in FIG. 5D, additional measurements can include estimating ejection fraction and volumes (at various points in the cardiac cycle and/or continuously through the full cardiac cycle) using the boundary location derived from speckle tracking to estimate the transition from blood pool to tissue. The resulting processed data, numerical measurements, bullseye plots and/or patient information are stored in a database and exported to a third party health care record management and reporting systems.

In a second specific example for visualization of a cardiac wall, ultrasound data is collected and processed in a manner similar to that described in the first specific example above. In this second specific example, measurements of the myocardium boundary can be utilized to alter the B-mode video loop to create an enhanced image with improved visualization of the cardiac wall, such as for use in wall motion scoring and/or to create a simulated view that resembles a contrast-agent injection study.

In a third specific example for assessment of atrial fibrillation, ultrasound data is collected over several cardiac cycles and spatially registered to one another. Because the timing of the cardiac cycles may differ as a result of arrhythmia, the data may then be averaged at representative time points (e.g., phases) across the several cardiac cycles to develop a single representative loop of data. The average loop of data may in turn be processed and measured similar to that described in the first and second examples, or any suitable manner.

In a fourth specific example for a study of cardio oncology, a series of ultrasound data is collected over several collection loops of cardiac cycles at different times or dates and are registered to one another. The ultrasound data in the fourth specific example is collected at a baseline measurement point and/or at different stages of a chemotherapy (or other) treatment. The data is then processed and synchronized in a manner similar to that described in the first specific example above. Measurements are made for displacements velocities, strain, strain rate, and/or other measurements in each of the loops and compared between various times or dates. Trends in peaks or continuous values of tissue properties may then be determined based on the series of data, for instance, across a baseline collection loop and one or more subsequent collection loops. Measurement plots are created and rendered for visualization showing these measurement values or comparisons through a series of video loops or series of still images depicting a trend.

As a person skilled in the art will recognize from the previous detailed description and from the figures and claims, modifications and changes can be made to the preferred embodiments of the invention without departing from the scope of this invention defined in the following claims.

We claim:

1. A method for acquiring and analyzing multiple image data loops comprising:
receiving a set of ultrasound data, characterizing a tissue, collected over a first collection loop and a second collection loop;
determining a tissue parameter distribution within the tissue based on the set of ultrasound data and multi-dimension speckle tracking, for both the first collection loop and the second collection loop;
producing a set of processed ultrasound data based on temporally synchronizing and spatially registering at least a portion of the set of ultrasound data from the first collection loop with a portion of the set of ultrasound data from the second collection loop;
receiving identification of at least one region of interest represented in the set of processed ultrasound data in the first collection loop and the second collection loop;
measuring a comparative characteristic, in the region of interest, within the first collection loop and the second collection loop; and
rendering at least one of the comparative characteristic and the tissue parameter distribution.

2. The method of claim 1, wherein receiving a set of ultrasound data collected over a first collection loop and a second collection loop comprises receiving a set of ultrasound data collected over a first collection loop comprising a subcycle of a first cardiac cycle and a second collection loop comprising the subcycle of a second cardiac cycle.

3. The method of claim 2, wherein the first cardiac cycle occurs during a rest state and wherein the second cardiac cycle occurs during a stress state.

4. The method of claim 2, wherein the first cardiac cycle occurs during a first phase of treatment and wherein the second cardiac cycle occurs during a second phase of treatment.

5. The method of claim 1, wherein receiving a set of ultrasound data collected over a first collection loop and a second collection loop comprises receiving a set of ultrasound data collected over a first collection loop from a first patient and a second collection loop from a second patient.

6. The method of claim 1, wherein determining a tissue parameter distribution within the tissue based on the set of ultrasound data and multi-dimension speckle tracking comprises determining a distribution of at least one of tissue displacement, tissue velocity, tissue strain, and tissue strain rate.

7. The method of claim 1, wherein temporally synchronizing and spatially registering at least a portion of the set of ultrasound data from the first collection loop with a portion of the set of ultrasound data from the second collection loop comprises temporally synchronizing a portion of the set of ultrasound data according to phases of a cardiac cycle.

8. The method of claim 1, wherein temporally synchronizing and spatially registering at least a portion of the set of ultrasound data from the first collection loop with a portion of the set of ultrasound data from the second collection loop comprises temporally synchronizing a portion of the set of ultrasound data using information from an additional signal.

9. The method of claim 8, wherein the additional signal is an electrocardiography signal.

10. The method of claim 1, wherein temporally synchronizing and spatially registering at least a portion of the set of ultrasound data from the first collection loop with a portion of the set of ultrasound data from the second collection loop comprises spatially registering at lest a portion of the set of ultrasound data by a defined tissue boundary.

11. The method of claim 1, further comprising analyzing at least one of B-mode features and tissue motion parameters from the set of ultrasound data.

12. The method of claim 1, wherein receiving identification of at least one region of interest represented in the set of processed ultrasound data in the first collection loop and the second collection loop comprises allowing a user to identify a region of interest at a user interface.

13. The method of claim 1, wherein receiving identification of at least one region of interest represented in the set of processed ultrasound data in the first collection loop and the second collection loop comprises automatically identifying a region of interest through boundary detection.

14. The method of claim 13, further comprising tracking an identified region of interest through multiple portions of the set of ultrasound data.

15. The method of claim 1, further comprising refining a region of interest based on at least one of morphological image processing and complementary data from another imagining modality.

16. The method of claim 1, further comprising receiving additional assessment data characterizing an aspect of the tissue.

17. The method of claim 16, wherein the additional assessment data comprises wall motion scores characterizing cardiac tissue motion.

18. The method of claim 1, wherein measuring a comparative characteristic, in the region of interest, within the first collection loop and the second collection loop comprises simultaneously measuring the comparative characteristic within the first collection loop and the second collection loop.

19. The method of claim 1, wherein measuring a comparative characteristic, in the region of interest, within the first collection loop and the second collection loop, comprises measuring at least one of tissue displacement, tissue velocity, tissue strain, tissue strain rate, and ejection fraction.

20. The method of claim 1, wherein measuring a comparative characteristic, in the region of interest, within the first collection loop and the second collection loop further comprises measuring a comparative characteristic and using the comparative characteristic to validate a visual assessment.

21. The method of claim 1, wherein rendering at least one of the comparative characteristic and the tissue parameter distribution comprises rendering at least one of still images, video loops, horseshoe graphics representing the myocardium, and bullseye mappings cardiac tissue.

22. The method of claim 1, further comprising storing at least one of the ultrasound data and measured comparative characteristics, exporting at least one of the ultrasound data and measured comparative characteristics, and analyzing at least one of the set of ultrasound data and a comparative characteristic for a relationship.

23. The method of claim 22, wherein analyzing at least one of the set of ultrasound data and a comparative characteristic for a relationship further comprises generating an analysis of a multiple patients.

24. The method of claim 1, further comprising:
receiving a set of ultrasound data, characterizing a tissue, collected over a third collection loop;
producing a set of processed ultrasound data based on temporally synchronizing and spatially registering at least a portion of the set of ultrasound data from the first collection loop with a portion of the set of ultrasound data from the second collection loop with a portion of the third collection loop;
receiving identification of at least one region of interest represented in the set of processed ultrasound data in the first collection loop, the second collection loop, and the third collection loop;
measuring a comparative characteristic, in the region of interest, within the first collection loop, the second collection loop, and the third collection loop third collection loop.

25. A system for acquiring and analyzing multiple image data loops comprising:
a processor comprising:
a first module configured to receive a set of ultrasound data, characterizing a tissue, collected over a first collection loop and a second collection loop,
a second module configured to determine a tissue parameter distribution within the tissue based on the set of ultrasound data and multi-dimension speckle tracking, and
a third module configured to receive identification of at least one region of interest represented in the set of ultrasound data in the first collection loop and the second collection loop;
an analysis engine configured to measure a comparative characteristic, in the region of interest, between the first collection loop and the second collection loop; and
a user interface, coupled to the processor and the analysis engine, and configured to render at least one of the comparative characteristic and the tissue parameter distribution.

26. The system of claim 25, further comprising an ultrasound scanner configured to acquire the set of ultrasound data.

27. The system of claim 25, wherein the system is further configured to couple to an electrocardiography module.

28. The system of claim 25, wherein the third module of the processor is configured to receive identification of at least one region of interest based on user interaction with the user interface.

29. The system of claim 25, wherein the processor further comprises a fourth module configured to temporally synchronize and spatially register at least a portion of the set of ultrasound data from the first collection loop with a portion of the set of ultrasound data from the second collection loop.
